(19) United States
(12) Patent Application Publication
(10) Pub. No.: US 2022/0031616 A1
(43) Pub. Date: Feb. 3, 2022

(54) Aerosolized CBD Liposomes for the treatment of Asthma and other pulmonary inflammatory disorders

(71) Applicants: Matthew Halpert, Pearland, TX (US); John Hartenbach, Irvine, CA (US)

(72) Inventors: Matthew Halpert, Pearland, TX (US); John Hartenbach, Irvine, CA (US)

(21) Appl. No.: 17/389,302

(22) Filed: Jul. 29, 2021

Related U.S. Application Data
(60) Provisional application No. 63/058,302, filed on Jul. 29, 2020.

(51) Int. Cl. A61K 9/127; A61K 31/05; A61K 45/06

(52) U.S. Cl. CPC A61K 9/127; A61K 45/06; A61K 31/05

(57) ABSTRACT

The present disclosure relates to the new compounds and improved treatment regimens that are effective in treating patients and minimizing the negative side effects previously associated with pulmonary care and treatment. Specifically, liposomal cannabinoids with or without other known treatments are used to provide a more effective and safer treatment for Asthma and/or pulmonary inflammation disorders or conditions in a diverse population.

### CROSS REFERENCE TO RELATED APPLICATION

This application claims the benefit of priority of U.S. Provisional Application Ser. No. 63/058,302, titled “Aerosolized CBD Liposomes for the treatment of Asthma and other pulmonary inflammatory disorders” filed Jul. 29, 2020, the entire content is incorporated herein by reference.

### BACKGROUND OF THE INVENTION

### Field of the Invention

The present invention generally relates to treatments for respiratory illness and breathing disorders and more specifically for Asthma and other pulmonary inflammatory conditions.

### Background Information

Lung disease is a major problem across the US and the world. The term lung disease refers to many disorders affecting the lungs such as asthma, chronic obstructive pulmonary disease (COPD), infections like coronavirus/Covid, Influenza, pneumonia and tuberculosis, lung cancer, and many other breathing problems. For example, about 25 million Americans suffer from Asthma, with about 10% of those being considered in “dire” conditions. About 4,000 US patients die each year from an Asthmatic attack, a relatively high number for something many consider to be completely benign. Looking globally, there are over 300 million people suffering from this condition.

The current treatment modalities are sub-par to say the least. This is largely in part due to a lack of understanding of what actually causes asthma. It is generally assumed that it is an aberrant immune response, perhaps an allergy, causing dangerous levels of inflammation in the airways. Thus, the treatments either only focus to assuage the symptoms (e.g albuterol, a non-specific bronchodilator that helps open up airways) or “throw the kitchen sink” at the accumulating immune cells (e.g. “steroids”). About 2 million Americans a year fail to respond to these treatments and end up hospitalized with no real treatment option (a.k.a. orphan disorder).

Thus, there is a need for improved treatment regimens that are effective in treating patients and minimizing the negative side effects. The embodiments of the present invention utilize liposomal cannabinoids with or without other known treatments to provide a more effective and safer treatment for lung diseases, including Asthma and/or pulmonary inflammation disorders or drug resistant treatment (such as steroid resistant asthma) or other pulmonary conditions in a diverse population.

### SUMMARY OF THE INVENTION

The present disclosure generally relates to the use of aerosolized cannabinoid liposomes for the treatment of lung disease conditions such as Asthma or other pulmonary inflammatory conditions or disorders.

One or more cannabinoid combinations are placed in a liposomal carrier and aerosolized to provide cannabinoid delivery directly within the pulmonary system. The one or more cannabinoids including but not limited to CBD, CBG, CBN, CBV, THC and any acids, salts or structural derivatives, for example THC would mean both delta 9 THC and delta 8 THC.

Additional embodiments would include the liposomal cannabinoid blend additionally including or being co-administered with known steroidal, or non-steroidal treatment products for either greater efficacy or for the ability of using a lower dose of the known steroidal or non-steroidal products.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: consists of FIGS. 1a, 1b, 1c, and 1d. FIG. 1 graphically displays Data from an airway mycosis-induced murine model of asthma. FIG. 1a schematically the study design for a mycosis induced murine model of asthma. FIG. 1b. graphically displays the changes in airway resistance to increasing doses of acetylcholine in challenged, non-challenged and treated or untreated mice. FIG. 1c graphically displays the differential cell counts for macrophages, eosinophils, neutrophils T cells and total cell count. FIG. 1d graphically displays T cell cytokine production.

FIG. 2: graphically displays the quantity of CBD as it is found further into the depths of the pulmonary system.

### DETAILED DESCRIPTION OF THE INVENTION

### Introduction

For the purpose of the present disclosure, the word “cannabinoid” refers to one or more cannabinoids or cannabinoid compounds or oils or extracts from plants (for example, hemp including hemp oil) that include one or a plurality of phytocannabinoids.

Cannabinoids are a group of cyclic molecules from Cannabis plants that activate cannabinoid receptors with the most well-known being CB1 and CB2 in cells. There are at least 85 different cannabinoids that can be isolated from Cannabis. Many cannabinoids produced by Cannabis plants, such as DELTA-9-Tetrahydrocannabinol (THC) and cannabidiol (CBD), have very low or no solubility in water. The most notable cannabinoids are THC and CBD. Additional examples include cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), and tetrahydrocannabivarinic acid (THCVA). Extracts of the Cannabis plant similarly include flavonoid compounds, terpenes, terpenoid, and synthetic, semisynthetic or highly purified versions of any such constituent.

The cannabinoids contemplated in the present invention include the derivatives and acids also associated with the listed cannabinoid, such as for THC the present invention include the various forms such as but not limited to: delta 9 THC, delta 8 THC, THCA and the like.

“Phytocannabinoids” are cannabinoids that originate from nature and can be found in the cannabis plant. The phytocannabinoids can be isolated from plants to produce a highly purified extract or can be reproduced synthetically.

“Highly purified cannabinoids” are defined as cannabinoids that have been extracted from the cannabis plant and purified to the extent that other cannabinoids and non-cannabinoid components that are co-extracted with the cannabinoids have been removed, such that the highly purified cannabinoid is greater than or equal to 95% (w/w) pure.

“Synthetic cannabinoids” are compounds that have a cannabinoid or cannabinoid-like structure and are manufactured using chemical means rather than by the plant.

Phytocannabinoids can be obtained as either the neutral (decarboxylated form) or the carboxylic acid form depending on the method used to extract the cannabinoids. For example, it is known that heating the carboxylic acid form will cause most of the carboxylic acid form to decarboxylate into the neutral form.

Several inflammatory situations in the airways are observed during chronic infections and are a result of accumulated and activated neutrophils, neutrophil extracellular traps (NETs), activated cells from the monocytic lineage, over-activated T cells, and/or a highly concentrated milieu of inflammatory cytokines, including but not limited to, TNFa, IL-6, IL-18, IL-4, IL-5, IL-13, IL-2, INFg, IL-1a, IL-1b, IL-23, IL-17, and IL-8. Additionally, several of these can become quickly and dangerously elevated during acute infections, and may be equally treatable as mentioned herein.

Lung inflammation can be characterized by an increase in active WBCs expressing a greater level of the activated conformation of LFA-1 compared to WBC's of a healthy subject without lung inflammation. These WBCs that have a greater level of the activated conformation of LFA-1 which cells can be identified in biological samples from a subject such as lung tissue, peripheral blood mononuclear cells (PBMCs) or a BAL sample, thus a clinician can determine whether a subject is in need of treatment for lung inflammation. A biological sample from a subject can be screened for the increased expression of certain cytokines (biomarkers) to determine whether the subject is in need of a treatment for lung inflammation. These cytokines/biomarkers include IL-4, IL-5, IL-9, IL-17F and IL-23alpha. Standard assays are known in the art to detect cytokines in biological samples, examples of biological samples include without limitation lung tissue, peripheral blood mononuclear cells (PBMCs) or a BAL sample.

Lung inflammation can be caused by many diseases and chronic conditions, such as asthma, chronic obstructive pulmonary disease (COPD), allergic bronchopulmonary aspergillosis, hypersensitivity pneumonia, eosinophilic pneumonia, emphysema, bronchitis, allergic bronchitis bronchiectasis, cystic fibrosis, tuberculosis, hypersensitivity pneumonitis, occupational asthma, sarcoid, reactive airway disease syndrome, interstitial lung disease, hyper-eosinophilic syndrome, rhinitis, sinusitis, exercise-induced asthma, pollution-induced asthma, cough variant asthma, parasitic lung disease, bacterial infections, respiratory syncytial virus (RSV) infection, parainfluenza virus (PIV) infection, rhinovirus (RV) infection and adenovirus infection. Many of the above described diseases and chronic disorders, cause an increase of WBCs expressing the activated conformation of LFA-1 to migrate and congregate in lung tissue and BAL and are suitable for treatment by the methods of the present invention.

Cystic fibrosis (CF) is a disease characterized by chronic inflammation and immune-mediated damage to the lung and airway, resulting in respiratory failure and death. Activated LFA-1 neutrophils responding to bacterial infection are predominantly responsible for the immune-mediated injury. Neutrophils generally play a role in the elimination of bacterial pathogens, however, in the case of CF, activated LFA-1 neutrophils are less immunologically effective. Asthma is a chronic condition that is also characterized by lung inflammation, whereby activated WBCs infiltrate into the airways and release inflammatory mediators which further cause bronchial epithelium damage. Allergic asthma is IgE mediated and involves initial exposure to an inhaled allergen and subsequent antigen presentation to T-helper type 2 lymphocytes, which secrete IL-4 and IL-13. Allergic asthma can be further characterized by airway inflammation in BAL and lung tissue, persistent Th2 response with increased cytokine production, progressive airway remodeling and bronchial hyperactivity.

COPD, or chronic obstructive pulmonary disease, is a progressive disease that makes it hard to breathe. COPD can cause coughing that produces large amounts of mucus (a slimy substance), wheezing, shortness of breath, chest tightness, and other symptoms. In COPD, less air flows in and out of the airways because of one or more of the following: the airways and air sacs lose their elastic quality; the walls between many of the air sacs are destroyed; the walls of the airways become thick and inflamed, and the airways make more mucus than usual, which can clog them.

In chronic bronchitis, the lining of the airways is constantly irritated and inflamed. This causes the lining to thicken. Lots of thick mucus forms in the airways, making it hard to breathe.

We have found that CBD can directly exert an anti-inflammatory role by doing things like reducing the production of TNFa and other inflammatory producing cytokines and reducing the activity of neutrophils. These, along with others such as eosinophils, are key players in dysregulated asthma. CBD has proven to exert a beneficial effect in asthmatic models. Previous studies utilized CBD in a smokable form as many people choose to smoke (a.k.a. inhale) CBD as a quicker (3-10 min) and ‘better’ form of increasing systemic bioavailability. Publications have shown that inhaled CBD can lead to a 30-65% systemic delivery, much higher than the ingestible amounts (Millar et al, 2018; Iffland, 2017; Lucas, 2018). However, in the case of asthma, this is exactly what we don't want. We want the CBD to stay in the pulmonary system to act on the recruited and activated immune cells.

Liposomes: A serious limitation of pulmonary delivery of drugs (and things like many cannabinoids such as CBD) is the poor water solubility. In addition, the thinness of the pulmonary epithelium results in short residence of the inhaled drug in the lung and potential for systemic (adverse) effects. Liposomes possess unique properties that make them suitable drug carriers. Liposomes are used to localize the action of inhaled drugs in the lung, improving the therapeutic outcome of the medication and reducing systemic adverse effects.

Dipalmitoylphosphatidylcholine liposomes given intratracheally in mice are taken up by pulmonary cells, and more than 50% of the phospholipid administered remained in the lung after 24 hours of administration. Thus, it is also more feasible to reach the ‘deeper’ layers of the bronchio-space by using liposomal carriers. Liposomes are used to retain the Cannabinoid compounds in the lungs longer than if not encapsulated.

Thus, embodiments of the present invention comprise of aerosolized cannabinoid products for the retention of the cannabinoid in the pulmonary space to treat the underlying and excessive inflammatory issues causing asthmatic-related symptoms. A specific cannabinoid contemplated is an aerosolized CBD-liposome product. Additional embodiments could include any of co-administration, co-formulation are the utilization of the Cannabinoid-liposome product with one or more other pulmonary treatment agents, such as steroids or non-steroidal drugs. A non-limiting example would include a cannabinoid-liposome product used in conjunction with a bronchodilator. When the cannabinoid-liposome product is used in coordination with a bronchodilator the treatment is more effective than a bronchodilator alone, because the bronchodilator only opens the airway up without treating the inflammation. The administration of the cannabinoid-liposome product can more effectively combat the inflammation and can reduce the need for steroids and excessive NSAIDs (all of which have negative side effects), and minimally give patients who have failed front-line therapies another option.

As used herein, the term “analog” refers to a compound that is structurally related to naturally occurring cannabinoids, but whose chemical and biological properties may differ from naturally occurring cannabinoids. In the present context, analog or analogs refer compounds that may not exhibit one or more undesirable side effects of a naturally occurring cannabinoid. In addition, analog refers to a compound that is derived from a naturally occurring cannabinoid by chemical, biological or a semi-synthetic transformation of the naturally occurring cannabinoid. Aspects of the present disclosure include liquid compositions of cannabinoids and their analogs. In addition, certain aspects also provide stable colloidal formulations that are manufactured by contacting a solution containing a cannabinoid, its analog, or both into a solvent such as water, with or without pharmaceutically acceptable buffers. A suitable solvent such as C1-C6 aliphatic alcohols or mixtures of water and alcohols, acetone or any water miscible organic solvent can be used to dissolve the cannabinoids.

In certain aspects, the inventive cannabinoid formulations are in the form of micelles or liposomes that encapsulate a cannabinoid or its analog within the membrane of the micelles or liposomes. Within the context of the present technology, the term “micelle” refers to an aggregate of surfactant molecules dispersed in a liquid colloid, while “liposome” refers to a vesicle composed of a mono or bilayer lipid.

Other drugs, and pharmaceutically acceptable carriers if present, may be in the lipophilic membrane or entrapped in the aqueous fluid that forms the core of the liposome. The entrapped cannabinoids contribute to the stability of the micelle/liposome membranes, such that the micelle/liposomes formulations may be used as an improved, fast, reliable and efficient system for the inhalable delivery of cannabinoids and/or additional drugs to subjects in need thereof. As used herein, the term “subject” refers to a mammal subject to the delivery of the compositions disclosed herein. Mammalian subjects include without limitation humans, dog, cat, horse or any other animal subject to the delivery of the disclosed compositions.

Unilamellar micelles or liposomes that are thermostable at temperatures greater than 50° C. can be used in the manufacture of cannabinoid formulations according to the present disclosure. These micelles or liposomes can be obtained by contacting a solution of a cannabinoid, its analog or both (a cannabinoid extract), with an aqueous solvent or an aqueous solution of a pharmaceutically active compound or drug. The mixing of the cannabinoid solution occurs in a manner suitable for the rapid dissolution of the cannabinoid solution in the aqueous solution. This can be accomplished through a variety of means including dilution, injection through a small orifice under pressure, and/or ultrasonic atomization.

For certain embodiments, the inventive composition is in the form of a concentrated, stable colloidal suspension that is obtained by infusing a solvent solution containing the cannabinoid extract or pure cannabinoids into a solvent such as water, with or without buffer. A stabilizing agent, for instance, a polymer or compounds selected from cellulose hyaluronic acid, polyvinyl pyrrolidone (PVP), alginate, chondroitin sulfate, poly gamma glutamic acid, gelatin, chitisin, corn starch and flour can be used to stabilize the micelle formulations.

According to one aspect of the present disclosure, the maximum final concentration of a cannabinoids or an analog of the cannabinoid in the micellar colloidal suspension is from about 1.0 mg/mL to about 100.0 mg/mL both values inclusive. For some embodiments, the concentration of a cannabinoid extract within liposomes can be greater than about 1.0 mg/mL, about 2.0 mg/mL, about 3.0 mg/mL, 4.0 mg/mL, about 5.0 mg/mL, about 6.0 mg/mL, about 7.0 mg/mL, about 8.0 mg/mL, about 9.0 mg/mL, about 10.0 mg/mL, about 15.0 mg/mL, about 20.0 mg/mL, about 25 mg/mL or about 30.0 mg/mL. In some aspects, a liposomal composition of the embodiments comprises about 20.0 mg/mL to about 30.0 mg/mL of cannabinoid.

Typical concentrations of cannabinoids within a liposomal suspension according to the present disclosure are about 50 mg/mL. For certain embodiments, the maximum final concentration of cannabinoids or an analog of the cannabinoid in the liposomal formulation is from about 10.0 mg/mL to about 300.0 mg/mL both values inclusive, for example, about 15.0 mg/mL, about 20.0 mg/mL, about 30.0 mg/mL, about 40.0 mg/mL, about 50.0 mg/mL, about 60.0 mg/mL, about 70.0 mg/mL, about 80.0 mg/mL, about 90.0 mg/mL, about 150.0 mg/mL, about 200.0 mg/mL, about 250.0 mg/mL, or about 300.0 mg/mL.

In some cases, the size of liposomes can range from about 0.01 μm to about 2.0 μm. For certain embodiments, the size of the spherical micelles is about 0.05 μm, about 0.1 μm, about 0.15 μm, 0.2 μm, 0.25 μm, 0.3 μm, 0.35 μm, 0.4 μm, 0.45 μm, 0.5 μm, 0.55 μm, 0.6 μm, 0.7 μm, 0.75 μm, 0.8 μm, 0.85 μm, 0.9 μm, about 0.95 μm, about 1.0 μm, 1.20 μm, 1.40 μm, 1.50 μm, 1.60 μm, 1.70 μm, 1.80 μm, 1.90 μm and 2.0 μm. For certain embodiments, liposomes that are about 0.04 μm, about 0.05 μm, about 0.06 μm, about 0.07 μm, about 0.08 μm, or about 0.09 μm are used to formulate compositions of the embodiments.

As used herein, cannabinoid compounds include without limitation cannabinol, cannabidiol, A9-tetrahydrocannabinol, A8-tetrahydrocannabinol, 11-hydroxy-tetrahydrocannabinol, 11-hydroxy-A9-tetrahydrocannabinol, levonantradol, A11-tetrahydrocannabinol, tetrahydrocannabivarin, dronabinol, amandamide, nabilone, any combination thereof, any natural or synthetic modification thereof, or any natural or synthetic molecule with a basic cannabinoid structure. In one preferred embodiment, the cannabinoid is tetrahydrocannabinol (THC).

Natural cannabinoid compounds used in the inventive compositions may be readily obtained from plant tissue, for example, trichones of the C. sativa plant, by suspending the tissue in an appropriate solvent to extract cannabinoid compounds and other tissue components. Analytical purification of such an extract provides pharmaceutical grade cannabinoid compounds. Cannabinoid compounds may also be extracted from plant tissue under supercritical conditions. Solvents used for supercritical extraction of cannabinoids include without limitation carbon dioxide, or other gases in isolation or combination with or without solvent modifiers, selected from ethanol, propanol, butanol, hexane, chloroform, dichloromethane, acetone, or any organic solvent capable of extracting cannabinoids, and alcohol-water mixtures, for instance water-ethanol or water-butanol mixtures.

In addition to natural cannabinoids, aspects of the present disclosure encompass synthetic cannabinoid compounds as well as cannabinoids and their analogs that are obtained using semi-synthetic protocols. The manufacture of cannabinoid compounds and their analogs using semi-synthetic means involves contacting an appropriate substrate with one of the cannabinoid synthase enzymes. In one example, tetrahydrocannabinolic acid (THCA) or its analogs can be manufactured semi-synthetically by contacting cannabigerolic acid (CBGA) or an appropriately substituted derivative of CBGA with THC synthase to obtain the corresponding THCA or THCA analog respectively. The compositions may also contain natural or synthetically modified cannabinoids.

The compositions according to the present disclosure have advantageous properties. For instance, micellar and liposomal compositions according to the present invention are stable at high temperatures (e.g. exceeding 50° C.), are stable to sonication, capable of carrying large payloads of cannabinoids as well as other drug suitable for use in combination therapy and can be stored for extended periods of time (e.g. greater than 20 weeks at 25° C.).

The compositions disclosed herein also exhibit superior inhalable delivery and release of cannabinoids from the micelle or liposomes used in the manufacture of the disclosed composition. The release of a cannabinoid from a liposome or micelle of the disclosed compositions can be modulated by changing the ratio of the concentration of lipid to the concentration of cannabinoid present in the liposome.

According to one embodiment, compositions of the present disclosure are used in the treatment of disease conditions. For instance, a composition of the cannabinoid extract, (cannabinoid, an analog of a cannabinoid, or both), can be administered to a patient or subject in need of treatment either alone or in combination with other compounds/drugs having similar or different biological activities.

For example, compositions of the present disclosure may be administered in a combination therapy, i.e., either simultaneously in single or separate dosage forms or in separate dosage forms within hours or days of each other. Examples of compounds/drugs used in such combination therapies include without limitation, chemotherapeutic agents, immunosuppressive agents, immunostimulatory, anti-pyretic, cytokines, opioids, cytokines, cytotoxic agents, nucleolytic compounds, radioactive isotopes, receptors, pro-drug activating enzymes, which may be naturally occurring or produced by recombinant methods, anti-inflammatory agents, antibiotics, protease inhibitors, growth factors, osteo-inductive factors and the like.

Prevention and/or treatment of infections can be achieved by the inclusion of antibiotics, as well as various antibacterial and antifungal agents, for example, paraben, chlorobutanol, phenol sorbic acid, and the like, in the compositions of the present disclosure.

One of ordinary skill will appreciate that effective amounts of the agents in the compositions used in the methods of the present disclosure can be determined empirically. It will be understood that, when administered to a human patient, the total daily usage of the composition of the present disclosure will be decided by the attending physician within the scope of sound medical judgment. The specific therapeutically effective dose level for any particular patient will depend upon a variety of factors: the type and degree of the response to be achieved; the activity of the specific composition employed; the age, body weight, general health, sex and diet of the patient; the duration of the treatment; drugs used in combination or coincidental with the method of the invention; and like factors well known in the medical arts.

Embodiments of the present disclosure thus generally described, will be understood more readily by reference to the following examples, which are provided by way of illustration only, and are not intended to be limit the scope of the present disclosure.

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Examples of Non-Limiting Scientific Studies Performed to Exemplify the Activities of the Embodied Methods of Treating and Compositions Used for Treatment of Various Physiological Conditions of Events.

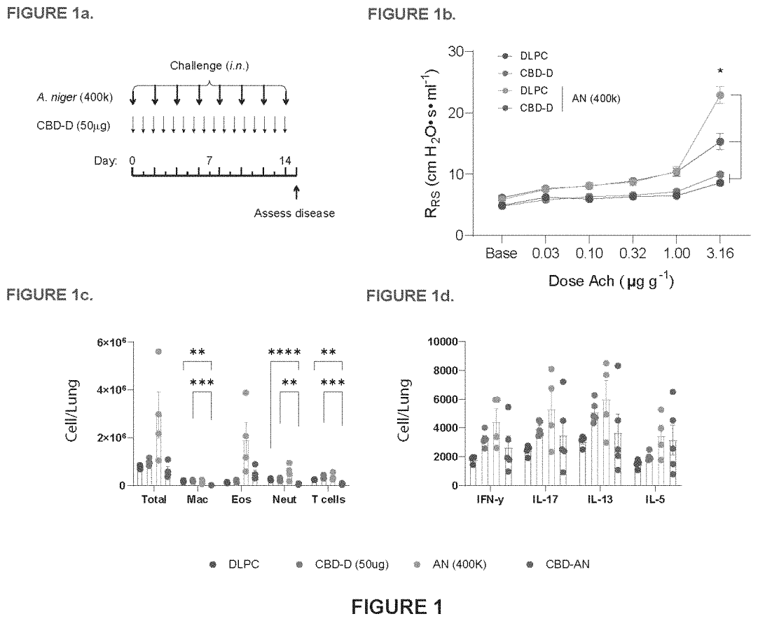

Data from an airway mycosis-induced murine model of asthma is demonstrated in FIG. 1. In FIG. 1a one set of mice were challenged with 4×10⁵ Aspergillus niger (AN) conidia every other day and treated every day with 50 mg CBD liposome formulation or vehicle (DLPC) formulation for 2 weeks and the another cohort of mice were not AN challenged but still received CBD or vehicle treatment daily. FIG. 1b. graphically displays the changes in airway resistance to increasing doses of acetylcholine. In the animals challenged with AN and treated with CBD there was a significant effect on the airway resistance as compared to vehicle control. FIG. 1c graphically displays the differential cell counts for macrophages, eosinophils, neutrophils T cells and total cell count. In all cases the CBD liposome treatment was shown to improve the physiological response to the AN challenge. CBD treated animals showed less total cells, less macrophages, less eosinophils less neutrophils and less T Cells, in the lung representing in some cases a complete protection from the AN challenge as compared to non-challenged controls. Additionally T cell cytokine production was graphically displayed in FIG. 1d. Again the CBD treated animals showed less cytokine elevation for IFN-γ, IL-17 IL-13 and IL-5 in the lung representing in some cases a complete protection from the AN challenge as compared to non-challenged controls.

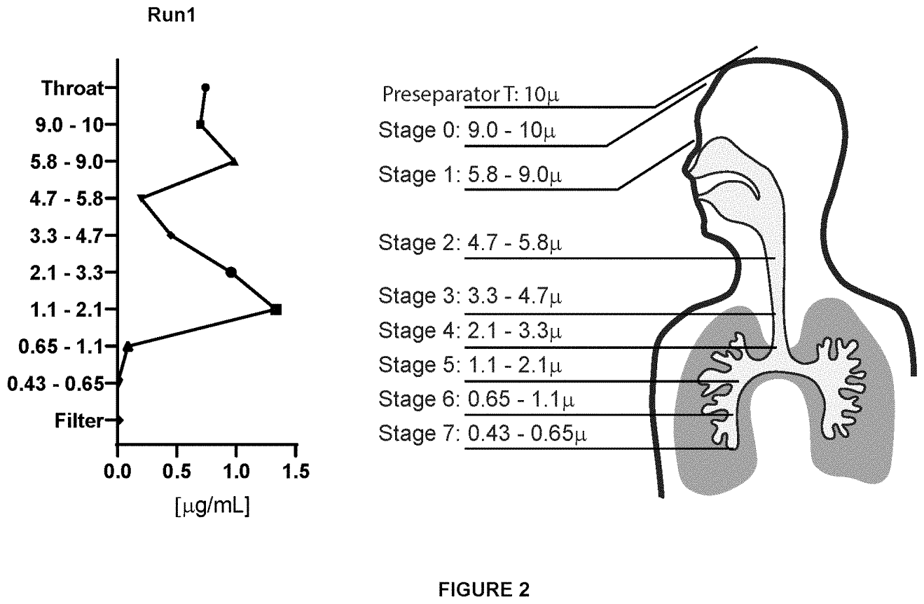

Data to confirm that the embodied CBD liposome formulations could reach in to various portions of the lung were derived from a CBD-DLPC (CBD-D) aerosol under constant flow of 5 L/minute. CBD presence was extracted off ACI plates and quantified by HPLC-MS the data is presented in FIG. 2 shows the quantity of CBD as it is found further into the depths of the pulmonary system. The data shows two peaks at around 6 and 2 microns, with most around the 2 micron size. The distribution of the aerosolized drug shows penetration all the way to the lower airway.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method for treating pulmonary inflammation disorders or conditions comprising a therapeutically effective amount of one or more endocannabinoids in a liposomal encapsulation.

2. The method of claim 1 wherein the one or more endocannabinoids is selected from CBD, delta 8 THC, delta 9 THC, CBG, CBN and CBV.

3. The method of claim 2 wherein one of the endocannabinoids is CBD.

4. A cannabinoid liposome formulation comprising: a liposome carrier and one or more cannabinoids in a formulation.

5. The method of claim 1 further comprising co-administering or co-formulating with one or more steroids, biologics, natural agents and NSAIDs with or without their own carrier or encapsulation formulations.
